(19) United States
(12) Patent Application Publication
(10) Pub. No.: US 2025/0059226 A1
(43) Pub. Date: Feb. 20, 2025

(54) Sulfinate glycosyl donor, method for preparing same, and use thereof

(71) Applicant: SICHUAN UNIVERSITY, Chengdu (CN)

(72) Inventors: Dawen NIU, Chengdu (CN); Xia ZHANG, Chengdu (CN); Chen ZHANG, Chengdu (CN); Hao ZUO, Chengdu (CN); Hongxin ZENG, Chengdu (CN); Yanjing LI, Chengdu (CN); Jiaxi WANG, Chengdu (CN)

(21) Appl. No.: 18/934,311

(22) Filed: Nov. 1, 2024

Related U.S. Application Data
(63) Continuation-in-part of application No. PCT/CN2022/118049, filed on Sep. 9, 2022.

(30) Foreign Application Priority Data
May 6, 2022 (CN) 202210488208.7

(51) Int. Cl. C07H 7/04; C07H 5/10; C07H 9/04; C07H 19/044

(52) U.S. Cl. CPC C07H 7/04; C07H 5/10; C07H 9/04; C07H 19/044

(57) ABSTRACT

A sulfinate glycosyl donor of formula I and a method for preparing same as well as use thereof in preparing a glycoside compound including a thioglycoside compound and a carbon glycoside compound are provided. The sulfinate glycosyl donor has a special sulfinate structure. When the sulfinate glycosyl donor is used as a raw material to prepare the glycoside compound, an additional initiator does not need to be added, such that the production cost is saved, the generation of byproducts is reduced, and the glycoside compound with a purity greater than 98% is obtained. The sulfinate glycosyl donor can be used in the preparation of the glycoside compound such as the thioglycoside compound and the carbon glycoside compound.

### FIELD OF THE INVENTION

The present invention relates to the field of pharmaceutical chemistry, and specifically to a sulfinate glycosyl donor, preparation methods therefor, and uses thereof.

### BACKGROUND OF THE INVENTION

Carbohydrates are important components of living organisms, including animals, plants, and microorganisms. Polysaccharides, oligosaccharides, and their glycoconjugates synthesized with proteins, esters, etc. involve all temporal and spatial processes of cells, especially multicellular lives. They act as information molecules and participate in various recognition processes of cells (transmitting biological information and participating in immune regulation of the body), and are closely related to various functions such as cell differentiation, fertilization, embryonic development, blood system, infection, aging, etc. In recent years, due to the significant physiological activity of carbohydrate compounds, they have increasingly attracted the research interest of chemists. Glycosides are important forms of sugars that exist in nature. They are widely present in living organisms, have special biological activities, and play important physiological functions. Glycosides are a class of very important compounds formed by the condensation of the semi-acetal hydroxyl of a sugar with a ligand via the loss of one molecule of water or other small molecule compounds, in which the sugar part is called the glycosyl, and the non-sugar part is called the ligand. According to the type of atom in the molecular structure of glycoside compounds, by which the ligand is linked to the carbon of the sugar ring, glycoside compounds can be divided into O-glycosides, N-glycosides, S-glycosides, and C-glycosides. Most of them exhibit good biological functions, such as glycosidase inhibition, antibacterial, antiviral, and anti-tumor activities.

The international patent application with application number WO2021013155A1 discloses an allylsulfone glycosyl donor and a method for preparing S-glycosides, O-glycosides, and C-glycosides using the allylsulfone glycosyl donor as starting materials, for example, a method for preparing aryl C-glycosides from the allylsulfone glycosyl donor and tetrafluoroborate pyridine salt (synthesis route being as follows). This method involves adding a glycosyl donor 3-1 (1.0 equiv), a glycosyl acceptor pyridinium tetrafluoroborate (2.0 equiv), a photosensitizer EosinY (Eosin Y, 0.025 equiv.), and an initiator sodium trifluoromethylsulfite (0.2 equiv.) to a catalytic reaction flask under nitrogen atmosphere, to which is added DMSO, and then stirring at room temperature for 8 h under the irradiation of Blue LED, to obtain aryl C-glycoside compound C-X.

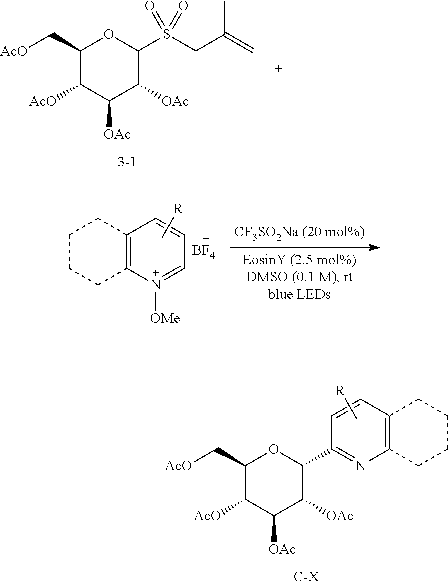

However, when the allylsulfone glycosyl donor is used to prepare aryl C-glycosides, an additional initiator sodium trifluoromethylsulfinate is required to induce the generation of sugar free radical intermediates, which has the following problems: 1. The additional initiator used increases the cost of the reaction; 2. It makes the reaction conditions more complex, and the compatibility worse; 3. It will generate additional by-products and make the separation of product more complex, thereby difficult to obtain high-purity of aryl C-glycosides.

Therefore, developing a new glycosyl donor that does not require the use of additional initiators in the preparation of glycosides is of great significance.

### Content of the Invention

The object of the present invention is to provide a sulfinate glycosyl donor with a novel structure, and a method preparing the same, as well as the use of said sulfinate glycosyl donor in the preparation of glycosides such as S-glycosides and C-glycosides (including aryl C-glycosides).

The present invention provides a glycosyl donor, or salts thereof, or stereoisomers thereof, or optical isomers thereof, and the structure of the glycosyl donor is as represented by formula W:

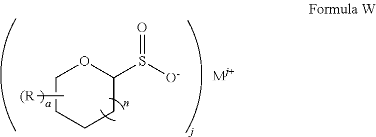

wherein, n is selected from 0 or 1;
a is selected from 3 or 4;
R is each independently selected from L₁Rₓ; or two adjacent R are linked to form a ring, while the other R is each independently selected from L₁Rₓ. The ring is either unsubstituted or substituted with one or more L₁Rₓ;
L₁ is selected from absence or C₁₋₂ alkylene, and Rₓ is selected from the group consisting of H, OH, C₁₋₆ alkyl, OAc, OBn, OR₈, NR₉R₁₀,

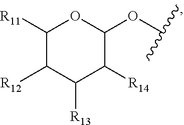

Ph, amino,

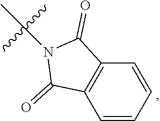 [SMILES: *N1C(=O)c2ccccc2C1=O]

and

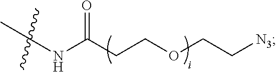

i is an integer selected from 0 to 6;
R₁₁, R₁₂, R₁₃, and R₁₄ are each independently selected from L₂R_{y}; L₂ is selected from absence or C₁₋₂ alkylene, and R_{y} is selected from the group consisting of H, OH, C₁₋₆ alkyl, OAc, OBn, OR₈, and NR₉R₁₀;
R₈ is selected from C₁₋₆ alkyl;
R₉ is selected from the group consisting of H, C₁₋₆ alkyl, Ac, and Bn;
R₁₀ is selected from the group consisting of H, C₁₋₆ alkyl, Ac, and Bn;
j is selected from 1, 2, or 3;
   M^{j+} is a j-valent cation.

Further, the structure of the glycosyl donor is as represented by formula I:

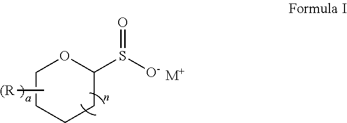

wherein, n is selected from 0 or 1;
a is selected from 3 or 4;
R is each independently selected from L₁Rₓ; or two adjacent R are linked to form a ring, while the other R is each independently selected from L₁Rₓ. The ring is either unsubstituted or substituted with one or more L₁Rₓ;
L₁ is selected from absence or C₁₋₂ alkylene, and Rₓ is selected from the group consisting of H, OH, C₁₋₆ alkyl, OAc, OBn, OR₈, NR₉R₁₀,

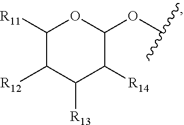

Ph, amino,

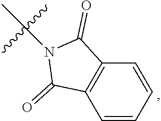 [SMILES: *N1C(=O)c2ccccc2C1=O]

and

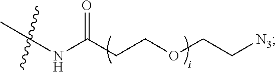

i is an integer selected from 0 to 6;
R₁₁, R₁₂, R₁₃, and R₁₄ are each independently selected from L₂R_{y}; L₂ is selected from absence or C₁₋₂ alkylene, and R_{y} is selected from the group consisting of H, OH, C₁₋₆ alkyl, OAc, OBn, OR₈, and NR₉R₁₀;
R₈ is selected from C₁₋₆ alkyl;
R₉ is selected from the group consisting of H, C₁₋₆ alkyl, Ac, and Bn;
R₁₀ is selected from the group consisting of H, C₁₋₆ alkyl, Ac, and Bn;
   M⁺ is a monovalent cation.

Further, the structure of the glycosyl donor is as represented by formula II:

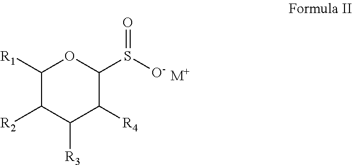

wherein, R₁, R₂, R₃, and R₄ are each independently selected from L₁Rₓ; alternatively, for R₁, R₂, R₃, and R₄, two adjacent groups are linked to form a ring, and the remaining two groups are each independently selected from L₁Rₓ. The ring is a 5-6-membered ring that is unsubstituted or substituted with one or more L₁Rₓ;
L₁ is selected from absence or methylene, and Rₓ is selected from the group consisting of H, OH, C₁₋₅ alkyl, OAc, OBn, OR₈, NR₉R₁₀,

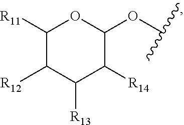

Ph, amino,

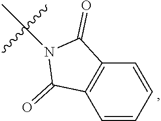 [SMILES: *N1C(=O)c2ccccc2C1=O]

and

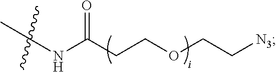

i is an integer selected from 0 to 4;
R₁₁, R₁₂, R₁₃, and R₁₄ are each independently selected from L₂R_{y}; L₂ is selected from absence or methylene, and R_{y} is selected from the group consisting of H, OH, C₁₋₅ alkyl, OAc, OBn, OR₈, and NR₉R₁₀;
R₈ is selected from C₁₋₅ alkyl;
R₉ is selected from the group consisting of H, C₁₋₅ alkyl, Ac, and Bn;
R₁₀ is selected from the group consisting of H, C₁₋₅ alkyl, Ac, and Bn;
M⁺ is a monovalent cation.

Further, the structure of the glycosyl donor is as represented by formula III:

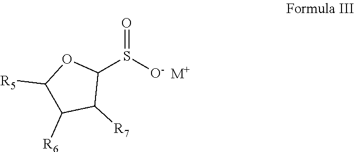

wherein, R₅, R₆, and R₇ are each independently selected from L₁Rₓ; alternatively, for R₅, R₆, and R₇, two adjacent groups are linked to form a ring, and the remaining group is L₁Rₓ. The ring is a 5-6-membered ring that is unsubstituted or substituted with one or more L₁Rₓ;
L₁ is selected from absence or methylene, and Rₓ is selected from the group consisting of H, OH, C₁₋₅ alkyl, OAc, OBn, OR₈, NR₉R₁₀,

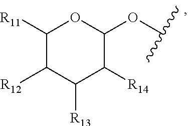

Ph, amino,

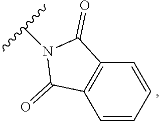 [SMILES: *N1C(=O)c2ccccc2C1=O]

and

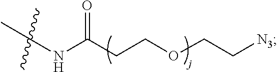

i is an integer selected from 0 to 4;
R₁₁, R₁₂, R₁₃, and R₁₄ are each independently selected from L₂R_{y}; L₂ is selected from absence or methylene, and R_{y} is selected from the group consisting of H, OH, C₁₋₅ alkyl, OAc, OBn, OR₈, and NR₉R₁₀;
R₈ is selected from C₁₋₅ alkyl;
R₉ is selected from the group consisting of H, C₁₋₅ alkyl, Ac, and Bn;
R₁₀ is selected from the group consisting of H, C₁₋₅ alkyl, Ac, and Bn;
   M⁺ is a monovalent cation.

Further, the 5-6-membered ring is a 5-6-membered saturated oxygen-containing heterocycle;
R₈ is selected from C₁₋₃ alkyl;
R₉ is selected from the group consisting of H, C₁₋₃ alkyl, Ac, and Bn;
R₁₀ is selected from the group consisting of H, C₁₋₃ alkyl, Ac, and Bn;
   M⁺ is selected from the group consisting of Na⁺, K⁺, and Li⁺.

Further, the structure of the glycosyl donor is selected from the group consisting of:

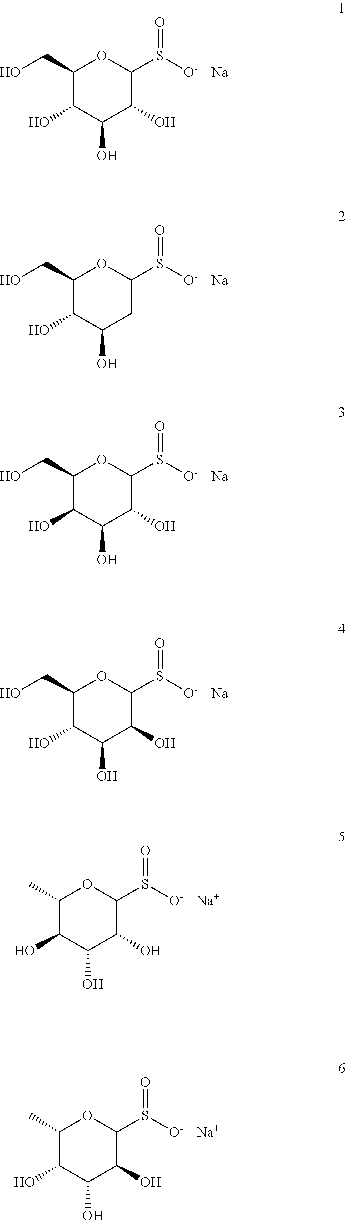

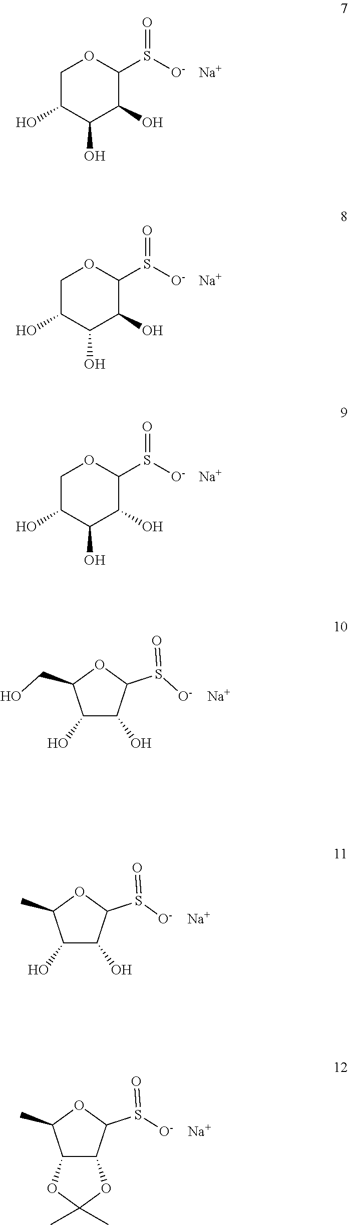

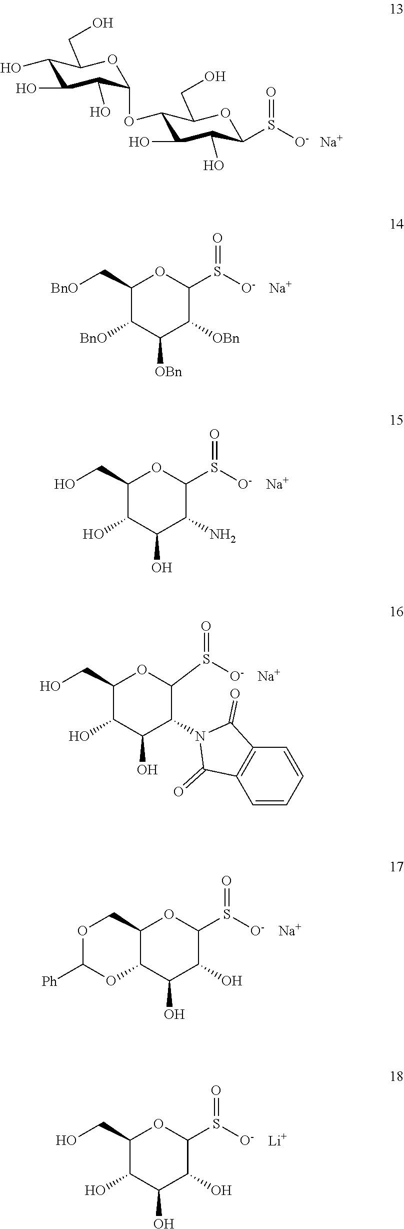

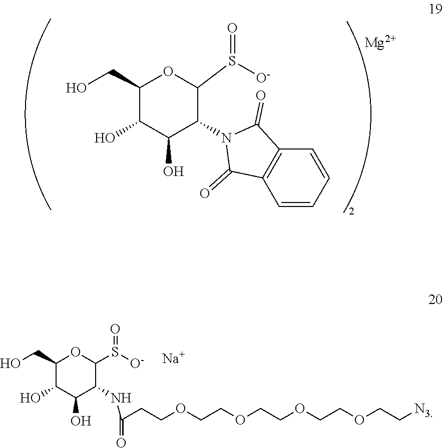

The present invention also provides a method for preparing the above glycosyl donor, which comprises the following steps:

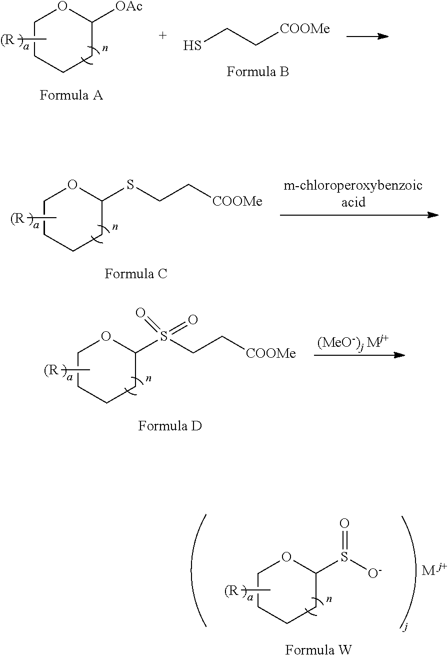

(1) The compound represented by formula A reacts with the compound represented by formula B, to obtain the compound represented by formula C;
(2) The compound represented by formula C reacts with m-chloroperoxybenzoic acid, to obtain the compound represented by formula D;
(3) The compound represented by formula D reacts with (MeO⁻)ⱼM^{j+}, to obtain the compound represented by formula W;
wherein n, a, R, M^{j+}, and j are as defined in the above.

Further, in step (1), the molar ratio of the compound represented by formula A to the compound represented by formula B is 1:(1.0-2.0), the reaction is carried out in the presence of Lewis acid, the molar ratio of the compound represented by formula A to Lewis acid is 1:(1.0-3.0), the reaction solvent is an organic solvent, the reaction temperature is room temperature, and the reaction time is 0.5-3 h;
in step (2), the molar ratio of the compound represented by formula A to m-chloroperoxybenzoic acid is 1:(2-4), the reaction solvent is an organic solvent, the reaction temperature is room temperature, and the reaction time is 0.5-3 h;
in step (3), the molar ratio of the compound represented by formula A to (MeO⁻)ⱼM^{j+} is 1:(0.5-1.5), the reaction solvent is an organic solvent, the reaction temperature is −5° C. to 5° C., and the reaction time is 1-3 h.

Further, in step (1), the molar ratio of the compound represented by formula A to the compound represented by formula B is 1:1.2, Lewis acid is BF₃·Et₂O, the molar ratio of the compound represented by formula A to Lewis acid is 1:2.0, the organic solvent is dichloromethane, and the reaction time is 1 h;
in step (2), the molar ratio of the compound represented by formula A to m-chloroperoxybenzoic acid is 1:3, the organic solvent is dichloromethane, and the reaction time is 1 h;
in step (3), the molar ratio of the compound represented by formula A to (MeO⁻)ⱼM^{j+} is 1:1, the organic solvent is methanol, the reaction temperature is 0° C., and the reaction time is 2 h.

The present invention also provides the use of the above glycosyl donor in the preparation of glycosides; the glycoside is preferably a S-glycoside or a C-glycoside.

Further, the C-glycoside comprises aryl C-glycosides.

The present invention also provides a method for preparing a C-glycoside, which comprises the following steps: the above glycosyl donor, the glycosyl acceptor represented by formula A-1, and a photosensitizer are added to a solvent, and then allowed to react under light, to obtain the C-glycoside represented by formula A-2;

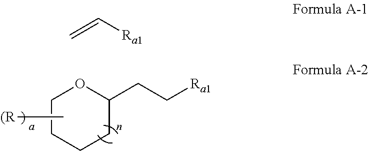

wherein R, a, and n are as defined in the above;
Rₐ₁ is selected from the group consisting of H, hydroxyl, C₁₋₆ alkyl, C₁₋₆ alkoxy, halogen, and the following moieties unsubstituted or substituted with one or more Rₐ₂ groups: 5-6-membered aryl, 5-6-membered heteroaryl, 3-8-membered saturated cycloalkyl, 3-8-membered saturated heterocyclyl; Rₐ₂ is selected from the group consisting of hydroxyl, C₁₋₆ alkyl, C₁₋₆ alkoxy, halogen, COORₐ₃, CORₐ₃; Rₐ₃ is selected from H and C₁₋₆ alkyl.

Further, the molar ratio of the above glycosyl donor, the glycosyl acceptor represented by formula A-1, and a photosensitizer is 1:(1-3):(5-20), and preferably 1:1.5:10;
the photosensitizer is selected from the group consisting of Ru (II) photosensitizer, eosin Y or a salt thereof, and said Ru (II) photosensitizer is preferably Ru(bpy)₃Cl₂·6H₂O, and the salt of eosin Y is preferably Eosin Y/Na⁺;
the solvent is an organic solvent; and preferably DMSO, DMF, DMA; and more preferably DMSO;
the reaction is carried out under a nitrogen atmosphere, the reaction temperature is room temperature, and the reaction time is 10-14 h;
said Rₐ₁ is

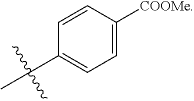 [SMILES: *c1ccc(C(=O)OC)cc1]

The present invention also provides a method for preparing a S-glycoside compound, which comprises the following steps: the above glycosyl donor, the glycosyl acceptor represented by formula C-1, and a photosensitizer are added to a solvent, and then allowed to react under light, to obtain the S-glycoside compound represented by formula C-2;

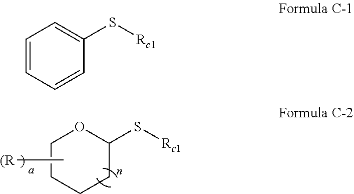

wherein R, a, and n are as defined in the above;
R_{c1} is selected from the group consisting of H, hydroxyl, C₁₋₆ alkyl, C₁₋₆ alkoxy, halogen, and the following moieties unsubstituted or substituted with one or more R_{c2} groups: 5-6-membered aryl, 5-6-membered heteroaryl, 3-8-membered saturated cycloalkyl, 3-8-membered saturated heterocyclyl; R_{c2} is selected from the group consisting of hydroxyl, C₁₋₆ alkyl, C₁₋₆ alkoxy, halogen, COOR_{c3}, COR_{c3}; R_{c3} is selected from H and C₁₋₆ alkyl.

Further, the molar ratio of the above glycosyl donor, the glycosyl acceptor represented by formula C-1, and a photosensitizer is 1:(1-3):(5-20), and preferably 1:1.5:10;

the photosensitizer is selected from the group consisting of Ru (II) photosensitizer, eosin Y or a salt thereof, and said Ru (II) photosensitizer is preferably Ru(bpy)₃Cl₂·6H₂O, and the salt of eosin Y is preferably Eosin Y/Na⁺;
the solvent is an organic solvent; and preferably DMSO, DMF, DMA; and more preferably DMSO;
the reaction is carried out under a nitrogen atmosphere, the reaction temperature is room temperature, and the reaction time is 10-14 h;
said R_{c1} is

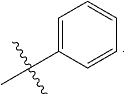 [SMILES: *c1ccccc1]

Further, the above light is performed by an LED lamp, with parameters of 10 W and 455 nm. Glycosyl donor refers to the starting material that contains a glycosidic bond during the synthesis of glycosides, or the starting material that contains an anomeric carbon joining in the reaction; and another starting material that reacts with it is called a glycosyl receptor.

The sulfinate glycosyl donor provided in the present invention has a novel structure containing a special sulfinate structure. The method for preparing the sulfinate glycosyl donor is simple, the reaction conditions are mild, and the yield is high, indicating the method is suitable for industrial production.

A variety of glycosides, including S-glycosides and C-glycosides (comprising aryl C-glycosides), were prepared using the sulfinate glycosyl donor of the present invention as the raw material. The preparation process is simple and does not require the addition of additional initiators, that can save production costs, reduce by-products, and provide glycosides with a purity of greater than 98%. The sulfinate glycosyl donor of the present invention has broad application prospects in the preparation of glycosides such as S-glycosides and C-glycosides (including aryl C-glycosides).

For the definition of terms used in the present invention: unless defined otherwise, the initial definition provided for the group or term herein applies to the group or term of the whole specification; for the terms that are not specifically defined herein, based on the disclosed content and context, they should have the same meaning as commonly understood by one of ordinary skill in the art to which the disclosure belongs.

The minimum and maximum values of carbon atom content in the hydrocarbon group are indicated by a prefix, for example, the prefix Ca-b alkyl indicates any alkyl having “a” to “b” carbon atoms. For example, C₁₋₈ alkyl means a straight or branched alkyl containing 1-8 carbon atoms. Similarly, C₁₋₆ alkoxy means a straight or branched alkoxy containing 1-6 carbon atoms.

For the groups of the present invention, Ac represents acetyl; Bn represents benzyl; Ph represents phenyl; Me represents methyl.

“m-CPBA” is m-chloro-peroxybenzoic acid.

“Salts” are acid and/or basic salts formed by compounds with inorganic and/or organic acids and/or bases, also including amphoteric salts (inner salts) as well as quaternary ammonium salts (e.g. alkylammonium salts). These salts can be directly obtained in the final isolation and purification of the compound, and can also be obtained by mixing the compound with a certain amount of acid or base (e.g., equivalent). These salts may form a precipitate in the solution and be collected by filtration, or recovered after evaporation of the solvent, or prepared by freeze-drying after reacting in an aqueous medium.

In the present invention, the salt may be hydrochloride, sulfate, citrate, benzenesulfonate, hydrobromide, hydrofluoride, phosphate, acetate, propionate, succinate, oxalate, malate, succinate, fumarate, maleate, tartrate or trifluoroacetate of the compound.

Obviously, based on the above content of the present invention, according to the common technical knowledge and the conventional means in the field, other various modifications, alternations, or changes can further be made, without department from the above basic technical spirits.

By following description of specific examples, the above content of the present invention is further illustrated. But it should not be construed that the scope of the above subject matter of the present invention is limited to the following examples. The techniques realized based on the above content of the present invention are all within the scope of the present invention.

### EXAMPLES

The starting materials and equipment used in the specific examples of the present invention are all known products, which are obtained by purchasing those commercially available.

The sodium sulfinate glycosyl donor of the present invention was prepared using the following synthetic route:

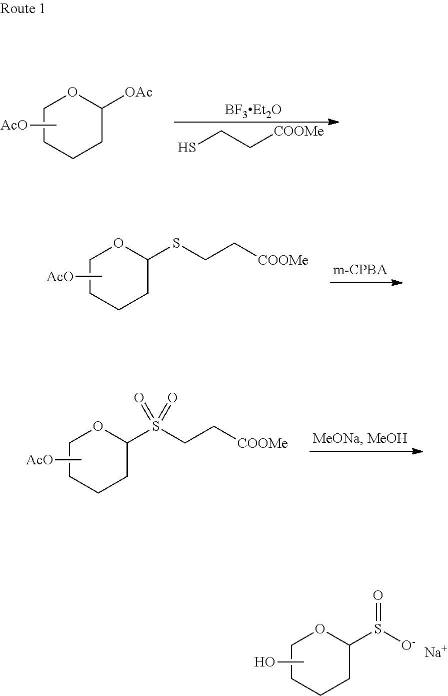

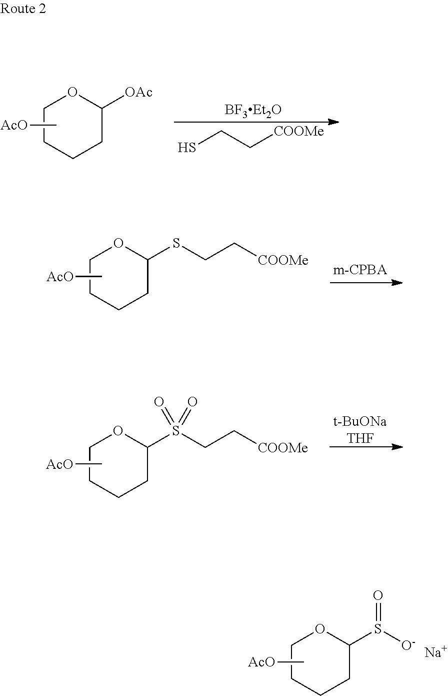

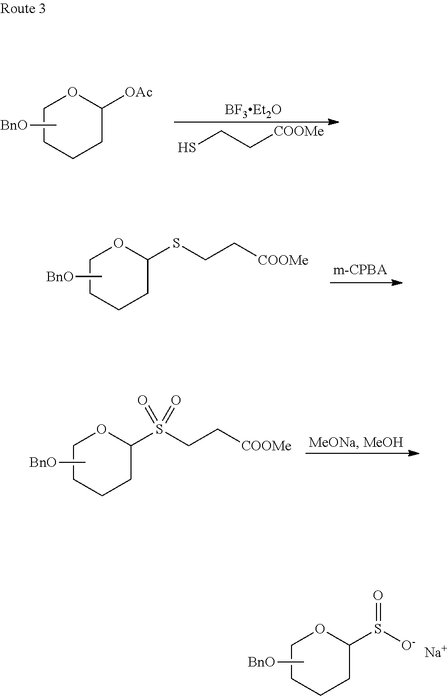

### Example 1: Preparation of Sodium Sulfinate Glycosyl Donors 1-20

### I. Preparation of Sodium Sulfinate Glycosyl Donor 1

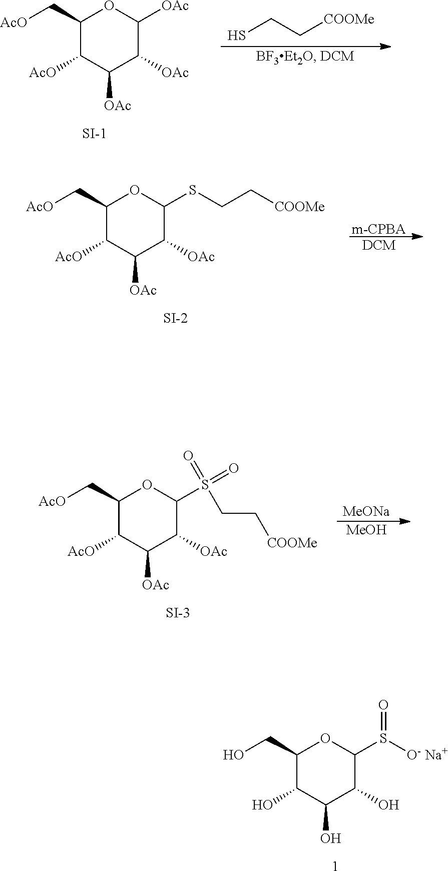

Specific procedures were as follows:

Step 1: To a 100 mL round bottom flask containing SI-1 (3.9 g, 10 mmol, 1.0 equiv) and 25 mL of CH₂Cl₂, were added methyl 3-mercaptopropionate (1.3 mL, 12 mmol, 1.2 equiv) and BF₃·Et₂O (2.5 mL, 20 mmol, 2.0 equiv) sequentially. The reaction solution was stirred at room temperature for 1 h, until SI-1 was completely disappeared by TLC detection, and then washed with saturated NaHCO₃ aqueous solution to be neutral. The organic layers were separated, washed with saline, dried over anhydrous Na₂SO₄, and then concentrated to obtain SI-2, which could be directly used for the next step without purification.

Step 2: SI-2 was dissolved in 20 mL of CH₂Cl₂ and then cooled at 0° C. m-CPBA (m-chloroperoxybenzoic acid, 6 g, 30 mmol, 3 equiv) was slowly added to the reaction solution under stirring. The mixed solution was stirred at room temperature for 1 h and filtered. The filtrate was washed with saturated NaHCO₃ solution until neutral, dried over anhydrous Na₂SO₄, and concentrated. Methyl tert-butyl ether was added to precipitate the solid, which was collected by filtration to obtain SI-3 as white solid.

Step 3: SI-3 was dissolved in 20 mL of MeOH at 0° C., to which was added MeONa (540 mg, 10 mmol, 1.0 equiv), and then the reaction was stirred at 0° C. for 2 h. TLC detection indicated that SI-3 was completely consumed before concentration. The residue was washed with absolute ethanol, and then the resultant solution was filtered to obtain white solid, namely sodium sulfinate glycosyl donor 1. The total yield for three steps was 85%.

### II. Preparation of Sodium Sulfinate Glycosyl Donors 2-20

Sodium sulfinate glycosyl donors 2-20 were prepared separately by referring to the above method for preparing sodium sulfinate glycosyl donor 1, with the only difference lying in that the raw material SI-1 was substituted with the corresponding starting materials, respectively.

The structure and characterization of sodium sulfinate glycosyl donors 1-20 are shown in Table 1. The total yield for three steps and the purity of sodium sulfinate glycosyl donors 1-20 are shown in Table 2.

TABLE 1
The structure and characterization of sodium sulfinate glycosyl donors 1-20.

| | | Starting materials | |
|---|---|---|---|
| No | Structure | corresponding to SI-1 | Structural characterization |
| 1 | 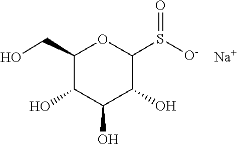 [SMILES: O=S([O-])C1O[C@H](CO)[C@@H](O)[C@H](O)[C@H]1O ; [Na+]] | 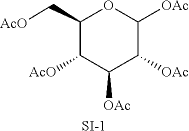 | ¹H NMR (400 MHz, CD₃OD) δ 3.85 (d, J = 12.9 Hz, 1H), 3.73 (t, J = 9.3 Hz, 1H), 3.66 (dd, J = 12.0, 5.2 Hz, 1H), 3.43 (t, J = 8.4 Hz, 1H), 3.35 (d, J = 9.7 Hz, 1H), 3.30 (d, J = 5.3 Hz, 2H); ¹³C NMR (101 MHz, D₂O) δ 92.68, 79.99, 77.10, 69.76, 69.16, 60.94. |
| 2 | 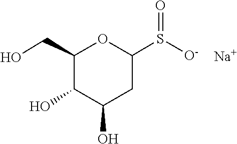 [SMILES: O=S([O-])C1C[C@@H](O)[C@H](O)[C@@H](CO)O1 ; [Na+]] | 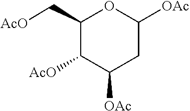 [SMILES: CC(=O)OC[C@H]1OC(OC(C)=O)C[C@@H](OC(C)=O)[C@@H]1OC(C)=O] | ¹³C NMR (101 MHz, D₂O) δ 96.29, 76.96, 70.67, 68.55, 60.78, 31.38. |
| 3 | 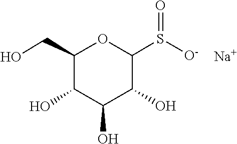 [SMILES: O=S([O-])C1O[C@H](CO)[C@@H](O)[C@H](O)[C@H]1O ; [Na+]] | 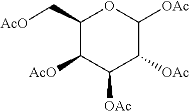 [SMILES: CC(=O)OC[C@H]1OC(OC(C)=O)[C@H](OC(C)=O)[C@@H](OC(C)=O)[C@H]1OC(C)=O] | ¹H NMR (400 MHz, CD₃OD) δ 3.99 (t, J = 9.5 Hz, 1H), 3.83 (dd, J = 11.7, 7.7 Hz, 1H), 3.78 (d, J = 3.3 Hz, 1H), 3.66 − 3.60 (m, 1H), 3.58 (q, J = 3.9 Hz, 1H), 3.53 (dd, J = 9.4, 3.4 Hz, 1H), 3.34 (d, J = 3.5 Hz, 1H). ¹³C NMR (101 MHz, D₂O) δ 93.72, 79.49, 73.96, |
| | | | 69.08, 67.06, 61.60. |
| 4 | 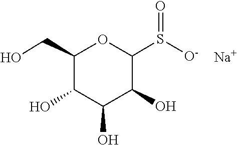 [SMILES: O=S([O-])C1O[C@H](CO)[C@@H](O)[C@H](O)[C@@H]1O ; [Na+]] | 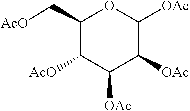 [SMILES: CC(=O)OC[C@H]1OC(OC(C)=O)[C@@H](OC(C)=O)[C@@H](OC(C)=O)[C@@H]1OC(C)=O] | ¹H NMR (400 MHz, D₂O) δ 4.22 (d, J = 3.0 Hz, 1H), 3.78 (m, 2H), 3.71 − 3.62 (m, 3H), 3.50 (d, J = 1.3 Hz, 1H); ¹³C NMR (101 MHz, D₂O) δ 100.65, 77.55, 71.25, 67.94, 66.20, 61.06 |
| 5 | 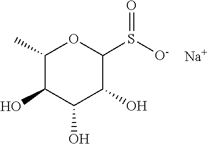 | 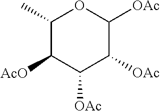 [SMILES: CC(=O)OC1O[C@@H](C)[C@H](OC(C)=O)[C@@H](OC(C)=O)[C@H]1OC(C)=O] | ¹H NMR (400 MHz, D₂O) δ 4.33 − 4.29 (m, 1H), 4.20 − 4.10 (m, 1H), 3.85 − 3.76 (m, 2H), 3.52 − 3.45 (m, 1H), 1.31 (d, J = 6.1, 3H). ¹³C NMR (101 MHz, D₂O) δ 90.32, 71.66, 69.90, 68.21, 57.41, 16.76. |
| 6 | 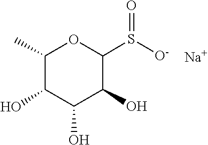 | 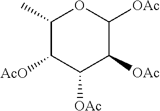 [SMILES: CC(=O)OC1O[C@@H](C)[C@@H](OC(C)=O)[C@@H](OC(C)=O)[C@@H]1OC(C)=O] | ¹H NMR (400 MHz, D₂O) δ 3.86 − 3.80 (m, 1H), 3.72 (d, J = 6.3 Hz, 1H), 3.62 (dt, J = 10.6, 2.9 Hz, 1H), 3.56 (td, J = 7.1, 1.9 Hz, 1H), 3.39 (dd, J = 9.7, 1.9 Hz, 1H), 1.22 − 1.17 (m, 3H); ¹³C NMR (101 MHz, D₂O) δ 93.75, 75.19, 74.10, 71.61, 66.57, 16.78. |
| 7 | 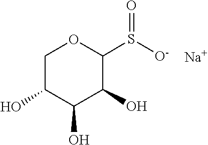 [SMILES: O=S([O-])C1OC[C@@H](O)[C@H](O)[C@@H]1O ; [Na+]] | 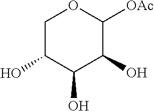 [SMILES: CC(=O)OC1OC[C@@H](O)[C@H](O)[C@@H]1O] | ¹³C NMR (101 MHz, D₂O) δ 99.50, 73.85, 70.79, 69.85, 68.38. |
| 8 | 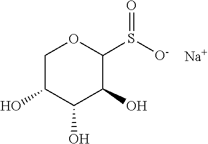 [SMILES: O=S([O-])C1OC[C@@H](O)[C@@H](O)[C@@H]1O ; [Na+]] | 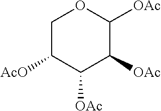 [SMILES: CC(=O)OC1OC[C@@H](OC(C)=O)[C@@H](OC(C)=O)[C@@H]1OC(C)=O] | Mixture of a and b□¹H NMR (400 MHz, D₂O) δ 4.37 (td, J = 3.9, 1.3 Hz, 0.5 H), 4.03 −3.83 (m, 3H), 3.75 − 3.54 (m, 2.5H); ¹³C NMR (101 MHz, D₂O) δ 96.71, 72.41, 71.81, 70.31, 68.44. |
| 9 | 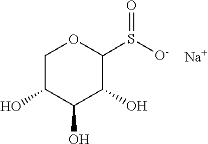 [SMILES: O=S([O-])C1OC[C@@H](O)[C@H](O)[C@H]1O ; [Na+]] | 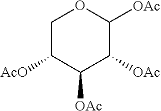 [SMILES: CC(=O)OC1OC[C@@H](OC(C)=O)[C@H](OC(C)=O)[C@H]1OC(C)=O] | ¹H NMR (400 MHz, D₂O) δ 3.98 (dd, J = 11.1, 5.3 Hz, 1H), 3.60 (t, J = 9.2 Hz, 1H), 3.52 (td, J = 9.7, 5.2 Hz, 1H), 3.42 (t, J = 9.2 Hz, 2H), 3.22 (t, J = 10.8 Hz, 1H); ¹³C NMR (101 MHz, D₂O) δ 93.61, 77.12, 69.54, 69.15, 68.86. |
| 10 | 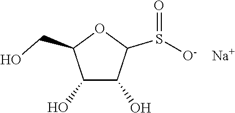 [SMILES: O=S([O-])C1O[C@H](CO)[C@@H](O)[C@H]1O ; [Na+]] | 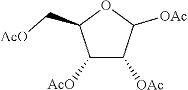 [SMILES: CC(=O)OC[C@H]1OC(OC(C)=O)[C@H](OC(C)=O)[C@@H]1OC(C)=O] | Mixture of α:β = 5:4, ¹H NMR (400 MHz, D₂O): δ 4.43 (t, J = 4.2 Hz, 0.43H), 4.29 (dd, J = 4.7, 2.1 Hz, 1H), 4.05 (dd, J = 8.4, 4.5 Hz, 0.55H), 4.00-3.91 (m, 2.46H), 3.88-3.68 (m, 2.94H), and 3.58 (td, J = |
| | | | 12.4, 4.7 Hz, 1.74H); |
| | | | ¹³C NMR (101 MHz, |
| | | | D₂O): δ 102.18, 100.63, |
| | | | 83.01, 82.53, 71.53, 71.30, |
| | | | 71.16, 70.70, 61.70, and |
| | | | 60.76., 2.89 (t, J = 6.9 Hz, |
| | | | 0.5H).¹³C NMR (101 MHz, |
| | | | D₂O) δ 102.09, 83.05, 71.29, |
| | | | 70.67, 61.67. |
| 11 | 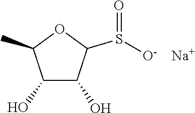 | 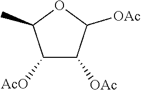 [SMILES: CC(=O)OC1O[C@H](C)[C@@H](OC(C)=O)[C@H]1OC(C)=O] | ¹H NMR (400 MHz, Methanol-d₄) δ 4.28 (d, J = 5.0 Hz, 1H), 3.96 (t, J = 6.3 Hz, 1H), 3.81 (t, J = 1.8 Hz, 1H), 3.73 − 3.65 (m, 1H), 1.21 (d, J = 6.3 Hz, 3H); ¹³C NMR (101 MHz, D₂O) δ 101.85, 78.88, 76.35, 70.51, |
| | | | 17.67. |
| 12 | 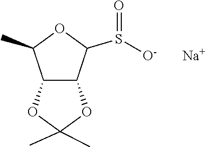 | 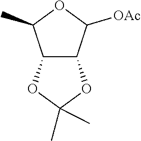 | ¹H NMR (400 MHz, CD₃OD) δ 5.01 (t, J = 4.5 Hz, 1H), 4.45 (d, J = 6.4 Hz, 1H), 4.20 (q, J = 7.1 Hz, 1H), 3.62 (dd, J = 4.4, 1.7 Hz, 1H), 1.46 (s, 3H), 1.30 (s, 3H), 1.15 (d, J = 5.1 Hz, 3H). |
| 13 | 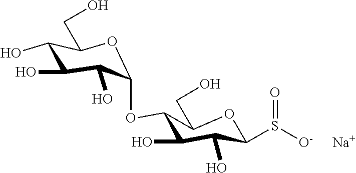 | 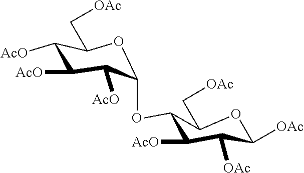 | ¹H NMR (400 MHz, D₂O) δ 5.32 (d, J = 3.8 Hz, 1H), 3.86 (d, J = 11.7 Hz, 1H), 3.81 − 3.44 (m, 11H), 3.33 (t, J = 9.4 Hz, 1H); ¹³C NMR (101 MHz, D₂O) δ 99.67, 92.47, 78.56, 77.51, 76.44, 72.83, 72.66, 71.73, 69.62, 69.30, 60.94, 60.43. |
| 14 | 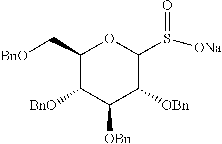 | 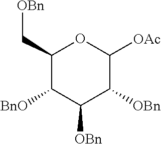 [SMILES: CC(=O)OC1O[C@H](COCc2ccccc2)[C@@H](OCc2ccccc2)[C@H](OCc2ccccc2)[C@H]1OCc1ccccc1] | ß only ¹H NMR (400 MHz, CDCl3 with 1%ₒ TFA): δ 7.36 − 7.27 (m, 14H), 7.26 − 7.22 (m, 4H), 7.15 − 7.06 (m, 2H), 4.93 (d, J = 11.1 Hz, 1H), 4.89 (d, J = 11.1 Hz, 1H), 4.81 (d, J = 10.7 Hz, 1H), 4.76 (d, J =11.1 Hz, |
| | | | 1H), 4.68 (d, J = 10.6 Hz, |
| | | | 1H), 4.44 (d, J = 11.3 Hz, |
| | | | 1H), 4.41 (d, J = 11.1 Hz, |
| | | | 1H), 4.36 (d,J = 11.2 Hz, |
| | | | 1H), 4.11 (d, J = 9.7 Hz, |
| | | | 1H), 3.85 (t, J = 8.8 Hz, 1H), |
| | | | 3.77 (t, J = 9.4 Hz, 1H), 3.67 |
| | | | (ddt, J = 7.9, 4.8, 2.9 Hz, |
| | | | 2H), 3.60 − 3.54 (m, 1H), |
| | | | and 3.51 (dd, J = 10.7, 5.4 |
| | | | Hz, 1H); |
| | | | ¹³C NMR (101 MHz, |
| | | | CDCl3 with 1%ₒ TFA): δ |
| | | | 161.1, 160.7, 160.2, 159.8, |
| | | | 137.0, 136.5, 136.3, 135.3, |
| | | | 129.0, 128.9, 128.7, 128.7, |
| | | | 128.6, 128.6, 128.5, 128.4, |
| | | | 128.3, 128.0, 118.7, 115. 9, |
| | | | 113.1, 110.2, 92.0, 85.7, |
| | | | 78.4, 77.3, 77.0, 76.8, 76.7, |
| | | | 76.1, 75.6, 75.4, 73.8, and |
| | | | 67.9. |
| 15 | 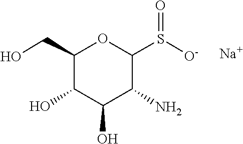 [SMILES: N[C@H]1C(S(=O)[O-])O[C@H](CO)[C@@H](O)[C@@H]1O ; [Na+]] | 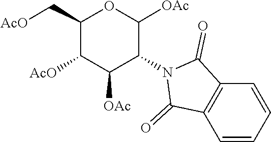 [SMILES: CC(=O)OC[C@H]1OC(OC(C)=O)[C@H](N2C(=O)c3ccccc3C2=O)[C@@H](OC(C)=O)[C@@H]1OC(C)=O] | ¹H NMR (400 MHz, D₂O) δ 3.92 (d, J = 13.0 Hz, 1H), 3.76 (dd, J = 12.5, 4.9 Hz, 1H), 3.71 (d, J = 10.3 Hz, 1H), 3.68 − 3.60 (m, 1H), 3.48 (ddd, J = 22.8, 17.9, 9.0 Hz, 2H), 3.39 − 3.31 (m, 1H). |
| 16 | 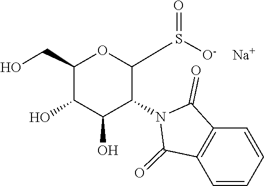 [SMILES: O=C1c2ccccc2C(=O)N1[C@H]1C(S(=O)[O-])O[C@H](CO)[C@@H](O)[C@@H]1O ; [Na+]] | 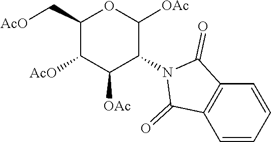 [SMILES: CC(=O)OC[C@H]1OC(OC(C)=O)[C@H](N2C(=O)c3ccccc3C2=O)[C@@H](OC(C)=O)[C@@H]1OC(C)=O] | ¹H NMR (400 MHz, MeOD) δ 7.84 (s, 2H), 7.77 (dd, J = 5.5, 3.1 Hz, 2H), 4.29 (dd, J = 6.7, 2.8 Hz, 2H), 4.13 (dd, J = 7.2, 3.3 Hz, 1H), 3.93 (dd, J = 12.1, 2.1 Hz, 1H), 3.73 − 3.67 (m, 1H), 3.46 (ddd, J = 9.3, 6.7, 2.2 Hz, 1H), 3.34 (s, 1H). |
| 17 | 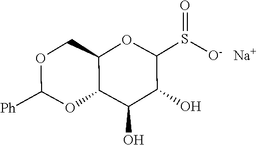 | 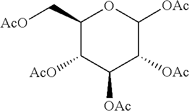 [SMILES: CC(=O)OC[C@H]1OC(OC(C)=O)[C@H](OC(C)=O)[C@@H](OC(C)=O)[C@@H]1OC(C)=O] | ¹H NMR (400 MHz, MeOD) δ 7.48 (dd, J = 6.8, 3.1 Hz, 2H), 7.41 (d, J = 2.5 Hz, 2H), 7.40 − 7.39 (m, 1H), 5.68 (s, 1H), 4.31 (td, 1H), 3.82 (t, J = 10.2 Hz, 1H), 3.78 − 3.72 (m, 2H), 3.60 (dqd, J = 9.3, 7.8, 7.3, 3.6 Hz, 3H). |
| 18 | 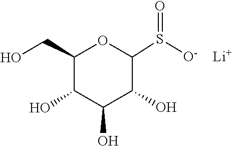 [SMILES: O=S([O-])C1O[C@H](CO)[C@@H](O)[C@H](O)[C@H]1O ; [Li+]] | 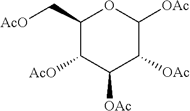 [SMILES: CC(=O)OC[C@H]1OC(OC(C)=O)[C@H](OC(C)=O)[C@@H](OC(C)=O)[C@@H]1OC(C)=O] | ¹H NMR (400 CD₃OD) δ 3.85 (d, J = 12.9 Hz, 1H), 3.73 (t, J = 9.3 Hz, 1H), 3.66 (dd, J = 12.0, 5.2 Hz, 1H), 3.43 (t, J = 8.4 Hz, 1H), 3.35 (d, J = 9.7 Hz, 1H), 3.30 (d, J = 5.3 Hz, 2H) |
| 19 | 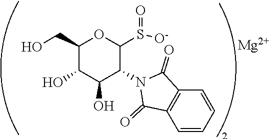 | 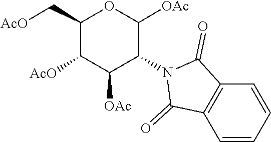 [SMILES: CC(=O)OC[C@H]1OC(OC(C)=O)[C@H](N2C(=O)c3ccccc3C2=O)[C@@H](OC(C)=O)[C@@H]1OC(C)=O] | ¹H NMR (400 MHz, CD₃OD) δ 7.83 − 7.76 (m, 1H), 7.55 − 7.50 (m, 1H), 7.47 (d, J = 4.5 Hz, 2H), 4.16 (t, J = 10.3 Hz, 1H), 3.90 (d, J = 12.1 Hz, 1H), 3.79 − 3.75 (m, 1H), 3.74 − 3.67 (m, 11H), 3.36 − 3.32 (m, 1H). |
| 20 | 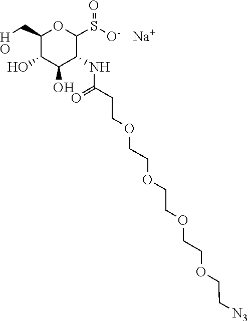 [SMILES: [N-]=[N+]=NCCOCCOCCOCCOCCC(=O)N[C@H]1C(S(=O)[O-])O[C@H](CO)[C@@H](O)[C@@H]1O ; [Na+]] | 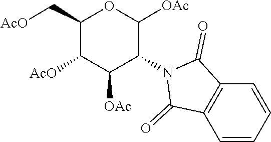 [SMILES: CC(=O)OC[C@H]1OC(OC(C)=O)[C@H](N2C(=O)c3ccccc3C2=O)[C@@H](OC(C)=O)[C@@H]1OC(C)=O] | ¹H NMR (400 MHz, D₂O) δ 3.94 (t, J = 10.3 Hz, 2H), 3.86 − 3.76 (m, 3H), 3.76 − 3.67 (m, 14H), 3.61 (d, J = 10.6 Hz, 2H), 3.54 − 3.50 (m, 2H), 3.49 − 3.42 (m, 2H), 2.58 (t, J = 6.2 Hz, 2H). |

TABLE 2
The total yield for three steps and the purity of sodium sulfinate glycosyl donors 1-20.

| | | Total yield for | |
|---|---|---|---|
| No. | Structure | three steps | Purity |
| 1 | 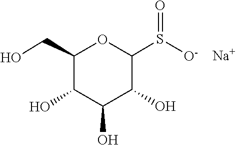 [SMILES: O=S([O-])C1O[C@H](CO)[C@@H](O)[C@H](O)[C@H]1O ; [Na+]] | 85% | 99% |
| 2 | 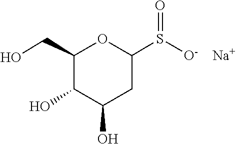 [SMILES: O=S([O-])C1C[C@@H](O)[C@H](O)[C@@H](CO)O1 ; [Na+]] | 77% | 95% |
| 3 | 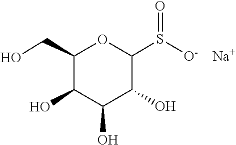 [SMILES: O=S([O-])C1O[C@H](CO)[C@H](O)[C@H](O)[C@H]1O ; [Na+]] | 87% | 98% |
| 4 | 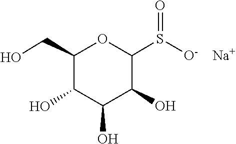 [SMILES: O=S([O-])C1O[C@H](CO)[C@@H](O)[C@H](O)[C@@H]1O ; [Na+]] | 75% | 98% |
| 5 | 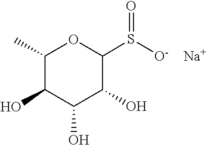 | 67% | 90% |
| 6 | 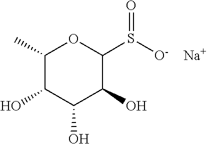 | 70% | 90% |
| 7 | 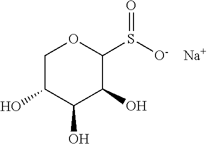 [SMILES: O=S([O-])C1OC[C@@H](O)[C@H](O)[C@@H]1O ; [Na+]] | 70% | 90% |
| 8 | 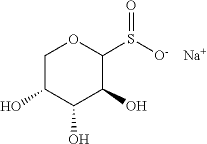 [SMILES: O=S([O-])C1OC[C@@H](O)[C@@H](O)[C@@H]1O ; [Na+]] | 70% | 90% |
| 9 | 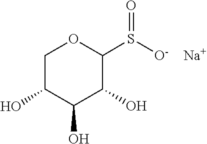 [SMILES: O=S([O-])C1OC[C@@H](O)[C@H](O)[C@H]1O ; [Na+]] | 70% | 90% |
| 10 | 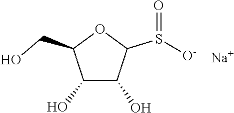 [SMILES: O=S([O-])C1O[C@H](CO)[C@@H](O)[C@H]1O ; [Na+]] | 65% | 85% |
| 11 | 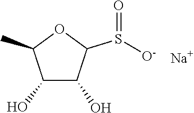 | 85% | 95% |
| 12 | 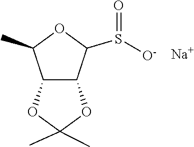 | 60% | 90% |
| 13 | 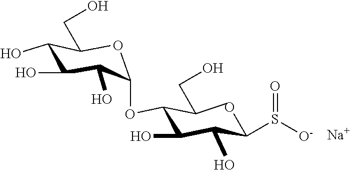 | 75% | 95% |
| 14 | 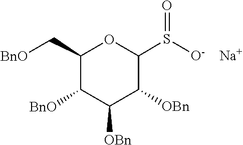 [SMILES: O=S([O-])C1O[C@H](COCc2ccccc2)[C@@H](OCc2ccccc2)[C@H](OCc2ccccc2)[C@H]1OCc1ccccc1 ; [Na+]] | 85% | 98% |
| 15 | 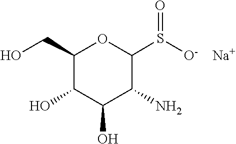 [SMILES: N[C@H]1C(S(=O)[O-])O[C@H](CO)[C@@H](O)[C@@H]1O ; [Na+]] | 82% | 98% |
| 16 | 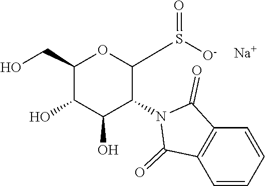 [SMILES: O=C1c2ccccc2C(=O)N1[C@H]1C(S(=O)[O-])O[C@H](CO)[C@@H](O)[C@@H]1O ; [Na+]] | 85% | 98% |
| 17 | 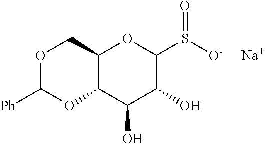 | 70% | 97% |
| 18 | 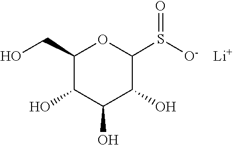 [SMILES: O=S([O-])C1O[C@H](CO)[C@@H](O)[C@H](O)[C@H]1O ; [Li+]] | 85% | 98% |
| 19 | 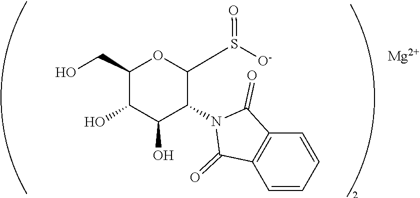 | 70% | 85% |
| 20 | 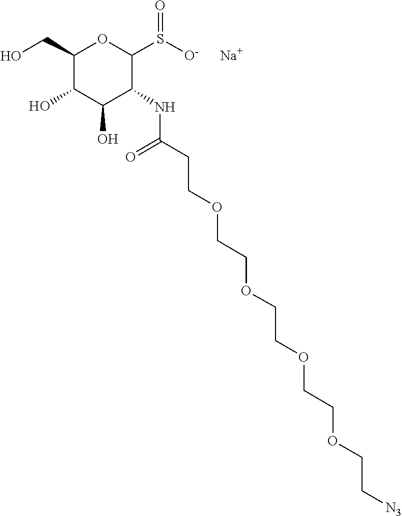 [SMILES: [N-]=[N+]=NCCOCCOCCOCCOCCC(=O)N[C@H]1C(S(=O)[O-])O[C@H](CO)[C@@H](O)[C@@H]1O ; [Na+]] | 57% | 98% |

### Example 2: Preparation of Non-Aryl C-Glycosides Using Sodium Sulfinate Glycosyl Donors as Raw Materials

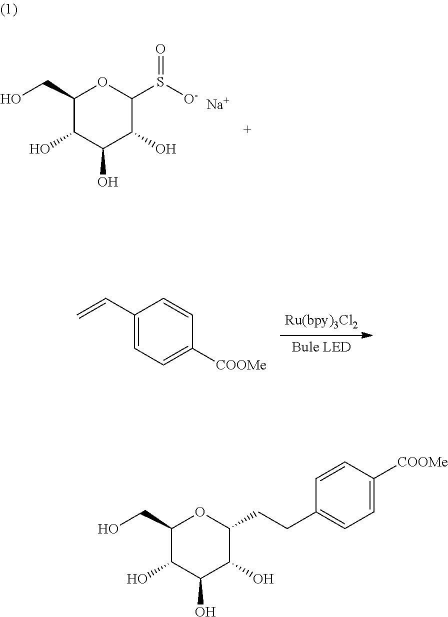 [SMILES: O=S([O-])C1O[C@H](CO)[C@@H](O)[C@H](O)[C@H]1O ; [Na+] ; C=Cc1ccc(C(=O)OC)cc1 ; COC(=O)c1ccc(CC[C@H]2O[C@H](CO)[C@@H](O)[C@H](O)[C@H]2O)cc1]

C-glycosides were synthesized according to the above route, and the specific procedures were as follows: Glycosyl donor 1 (0.2 mmol), 4-methoxycarbonylstyrene (0.3 mmol), photosensitizer Ru(bpy)₃Cl₂·6H₂O (1 mol %), and DMSO (0.5 mL) were weighed, transferred into a vial with a spiral cap and a magnetic stirring bar, and then mixed. The vial was filled with N₂ and sealed with a Teflon cap. The mixture was stirred at room temperature under a 10 W, 455 nm LED light for 12 h to complete the reaction.

The reaction solution was freeze-dried and subjected to column chromatography, to obtain C-glycoside compounds as pure α-configuration, with a yield of 90% and a purity of >98%. The structural characterization was as follows:

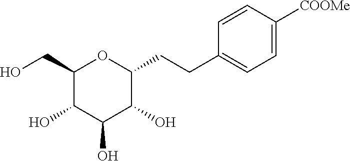 [SMILES: COC(=O)c1ccc(CC[C@H]2O[C@H](CO)[C@@H](O)[C@H](O)[C@H]2O)cc1]

¹H NMR (400 MHZ, DMSO-d₆) δ 7.88 (d, J=7.9 Hz, 2H), 7.38 (d, J=7.9 Hz, 2H), 4.89 (d, J=4.4 Hz, 1H), 4.81 (d, J=5.3 Hz, 2H), 4.48 (t, J=6.0 Hz, 1H), 3.84 (s, 3H), 3.73-3.61 (m, 2H), 3.46-3.41 (m, 1H), 3.32-3.28 (m, 1H), 3.02 (td, J=8.7, 5.5 Hz, 1H), 2.80 (ddd, J=14.2, 9.5, 5.2 Hz, 1H).

(2) By reference to the above method, corresponding C-glycoside compounds were respectively prepared by substituting glycosyl donor 1 with glycosyl donors 2-20.

### Example 3: Preparation of Aryl C-Glycoside Compounds Using Sodium Sulfinate Glycosyl Donors as Raw Materials

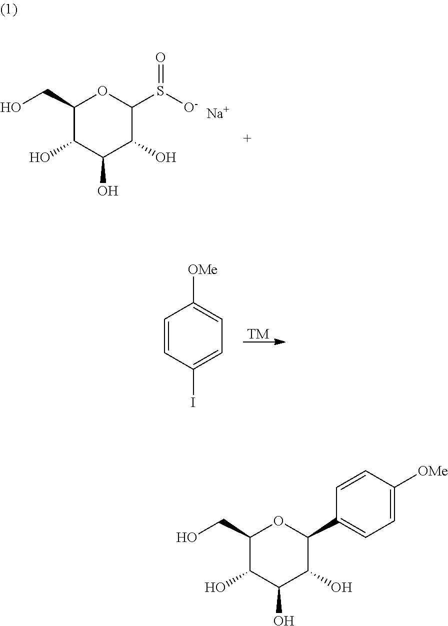

Aryl C-glycoside compounds were synthesized according to the above route, and the specific procedures were as follows: Glycosyl donor 1 (0.15 mmol), 4-methoxyiodobenzene (0.1 mmol), Ru(bpy)₃Cl₂·6H₂O (0.8 mg, 1 mol %), NiBr₂·DME (3.1 mg, 10 mol %), diOMebpy (4,4′-dimethoxy-2,2′-bipyridine, 2.6 mg, 12 mol %), TMG (tetramethylguanidine, 12.5 μL, 0.2 mmol) and DMSO (0.5 mL) were weighed, placed into a vial with a spiral cap and a magnetic stirring bar, and then mixed. The vial was filled with N₂ and sealed with a Teflon cap. The mixture was stirred at room temperature under a 10 W, 455 nm LED light for 12 h, and then the reaction was completed.

The reaction solution was freeze-dried, and the residue was subjected to column chromatography (C₁₈, H₂O/MeCN=13:1), to obtain aryl C-glycoside as pure β-configuration, with a yield of 67% and a purity of >98%. The structural characterization was as follows:

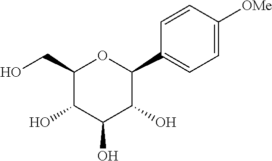 [SMILES: COc1ccc([C@@H]2O[C@H](CO)[C@@H](O)[C@H](O)[C@H]2O)cc1]

¹H NMR (400 MHz, DMSO-d₆) δ 7.25 (d, J=8.1 Hz, 2H), 6.86 (d, J=8.2 Hz, 2H), 4.98-4.83 (m, 2H), 4.70 (d, J=5.7 Hz, 1H), 4.42 (t, J=5.8 Hz, 1H), 3.95 (d, J=9.3 Hz, 1H), 3.82-3.59 (m, 4H), 3.44 (dt, J=11.8, 5.9 Hz, 1H), 3.30-3.08 (m, 4H); ¹³C NMR (101 MHZ, DMSO-d₆) δ 158.59, 132.50, 128.97, 113.07, 81.16, 81.04, 78.49, 74.67, 70.45, 61.48, 55.05; HRMS (DART-TOF) calculated for C₁₃H₁₈NaO₆⁺ [M+Na]⁺ m/z 293.0996, found 293.0995.

(2) By referring to the above method, corresponding aryl C-glycosides were respectively prepared by substituting glycosyl donor 1 with glycosyl donors 2-20.

### Example 4: Preparation of S-Glycoside Compounds Using Sodium Sulfinate Glycosyl Donors as Raw Materials

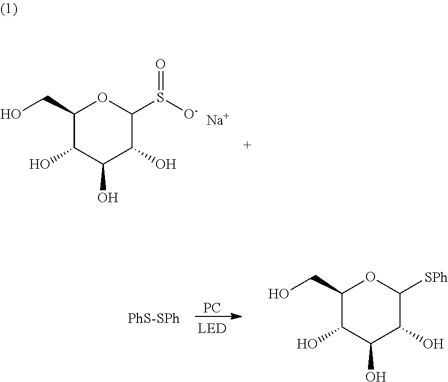

S-glycoside compounds were synthesized according to the above route, and the specific procedures were as follows: Glycosyl donor 1 (0.2 mmol), phenyl disulfide (0.3 mmol), Ru(bpy)₃Cl₂·6H₂O (1 mol %), and DMSO (0.5 mL) were weighed, placed into a vial with a spiral cap and a magnetic stirring bar, and then mixed. The vial was filled with N₂ and sealed with a Teflon cap. The mixture was stirred at room temperature under a 10 W, 455 nm LED light for 12 h, and then the reaction was completed.

The reaction solution was freeze-dried, and then subjected to column chromatography, to obtain the glycoside as pure α-configuration, with a yield of 95% and a purity of >98%. The structural characterization was as follows:

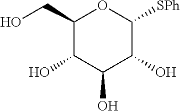

¹H NMR (400 MHZ, DMSO-d₆) δ 7.49 (d, J=7.1 Hz, 2H), 7.31 (t, J=7.4 Hz, 2H), 7.24 (t, J=7.3 Hz, 1H), 5.49 (d, J=5.3 Hz, 1H), 5.38 (d, J=4.3 Hz, 1H), 5.02 (d, J=5.4 Hz, 2H), 4.48 (t, J=5.9 Hz, 1H), 3.85 (ddd, J=10.0, 5.0, 2.3 Hz, 1H), 3.62-3.47 (m, 3H), 3.18 (dd, J=9.3, 5.5 Hz, 1H), 2.50 (t, J=1.9 Hz, 1H).

(2)

By referring to the above method, corresponding S-glycoside compounds were respectively prepared by substituting glycosyl donor 1 with glycosyl donors 2-20.

In summary, the present invention had provided a sulfinate glycosyl donor represented by formula I, and a method preparing the same, as well as the use of the sulfinate glycosyl donor represented by formula I in the preparation of glycosides such as S-glycosides and C-glycosides. The sulfinate glycosyl donor provided in the present invention had a novel structure containing a special sulfinate structure. The method for preparing the sulfinate glycosyl donor was simple, the reaction conditions were mild, and the yield was high, indicating the method was suitable for industrial production. If the sulfinate glycosyl donor of the present invention, as the raw material, was used to prepare glycosides, the addition of additional initiators was not required, that could save production costs, reduce by-products, and provide glycosides with a purity of >98%. The sulfinate glycosyl donor of the present invention had broad application prospects in the preparation of glycosides such as S-glycosides and C-glycosides (including aryl C-glycosides).

1. A glycosyl donor, or salts thereof, or stereoisomers thereof, or optical isomers thereof, characterized in that the structure of the glycosyl donor is as represented by formula W: 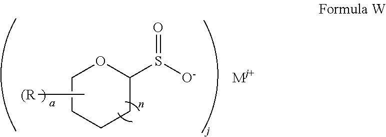
wherein, n is selected from 0 or 1;
a is selected from 3 or 4;
R is each independently selected from L₁Rₓ; or two adjacent R are linked to form a ring, while the other R is each independently selected from L₁Rₓ. The ring is either unsubstituted or substituted with one or more L₁Rₓ;
L₁ is selected from absence or C₁₋₂ alkylene, and Rₓ is selected from the group consisting of H, OH, C₁₋₆ alkyl, OAc, OBn, OR₈, NR₉R₁₀, 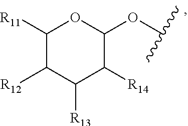
Ph, amino, 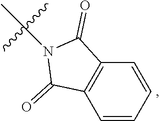 [SMILES: *N1C(=O)c2ccccc2C1=O]
and 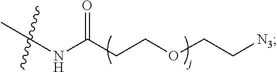
i is an integer selected from 0 to 6;
R₁₁, R₁₂, R₁₃, and R₁₄ are each independently selected from L₂R_{y}; L₂ is selected from absence or C₁₋₂ alkylene, and R_{y} is selected from the group consisting of H, OH, C₁₋₆ alkyl, OAc, OBn, OR₈, and NR₉R₁₀;
R₈ is selected from C₁₋₆ alkyl;
R₉ is selected from the group consisting of H, C₁₋₆ alkyl, Ac, and Bn;
R₁₀ is selected from the group consisting of H, C₁₋₆ alkyl, Ac, and Bn;
j is selected from 1, 2, or 3;
M^{j+} is a j-valent cation.

2. The glycosyl donor according to claim 1, or salts thereof, or stereoisomers thereof, or optical isomers thereof, characterized in that the structure of the glycosyl donor is as represented by formula I: 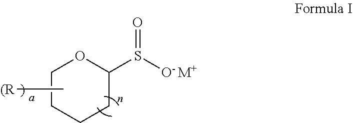
wherein, n is selected from 0 or 1;
a is selected from 3 or 4;
R is each independently selected from L₁Rₓ; or two adjacent R are linked to form a ring, while the other R is each independently selected from L₁Rₓ. The ring is either unsubstituted or substituted with one or more L₁Rₓ;
L₁ is selected from absence or C₁₋₂ alkylene, and Rₓ is selected from the group consisting of H, OH, C₁₋₆ alkyl, OAc, OBn, OR₈, NR₉R₁₀, 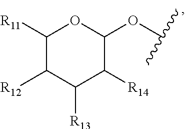
Ph, amino, 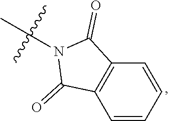 [SMILES: *N1C(=O)c2ccccc2C1=O]
and 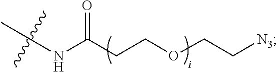
i is an integer selected from 0 to 6;
R₁₁, R₁₂, R₁₃, and R₁₄ are each independently selected from L₂R_{y}; L₂ is selected from absence or C₁₋₂ alkylene, and R_{y} is selected from the group consisting of H, OH, C₁₋₆ alkyl, OAc, OBn, OR₈, and NR₉R₁₀;
R₈ is selected from C₁₋₆ alkyl;
R₉ is selected from the group consisting of H, C₁₋₆ alkyl, Ac, and Bn;
R₁₀ is selected from the group consisting of H, C₁₋₆ alkyl, Ac, and Bn;
M⁺ is a monovalent cation.

3. The glycosyl donor according to claim 2, or salts thereof, or stereoisomers thereof, or optical isomers thereof, characterized in that the structure of the glycosyl donor is as represented by formula II: 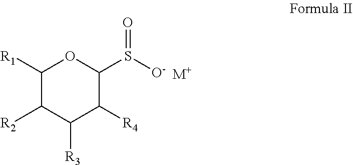
wherein, R₁, R₂, R₃, and R₄ are each independently selected from L₁Rₓ; alternatively, for R₁, R₂, R₃, and R₄, two adjacent groups are linked to form a ring, and the remaining two groups are each independently selected from L₁Rₓ. The ring is a 5-6-membered ring which is unsubstituted or substituted with one or more L₁Rₓ;
L₁ is selected from absence or methylene, and Rₓ is selected from the group consisting of H, OH, C₁₋₅ alkyl, OAc, OBn, OR₈, NR₉R₁₀, 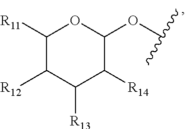
Ph, amino, 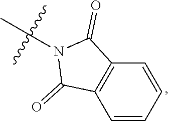 [SMILES: *N1C(=O)c2ccccc2C1=O]
and 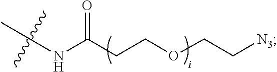
i is an integer selected from 0 to 4;
R₁₁, R₁₂, R₁₃, and R₁₄ are each independently selected from L₂R_{y}; L₂ is selected from absence or methylene, and R_{y} is selected from the group consisting of H, OH, C₁₋₅ alkyl, OAc, OBn, OR₈, and NR₉R₁₀;
R₈ is selected from C₁₋₅ alkyl;
R₉ is selected from the group consisting of H, C₁₋₅ alkyl, Ac, and Bn;
R₁₀ is selected from the group consisting of H, C₁₋₅ alkyl, Ac, and Bn;
M⁺ is a monovalent cation.

4. The glycosyl donor according to claim 2, or salts thereof, or stereoisomers thereof, or optical isomers thereof, characterized in that the structure of the glycosyl donor is as represented by formula III: 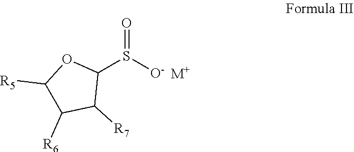
wherein, R₅, R₆, and R₇ are each independently selected from L₁Rₓ; alternatively, for R₅, R₆, and R₇, two adjacent groups are linked to form a ring, and the remaining group is L₁Rₓ. The ring is a 5-6-membered ring that is unsubstituted or substituted with one or more L₁Rₓ;
L₁ is selected from absence or methylene, and Rₓ is selected from the group consisting of H, OH, C₁₋₅ alkyl, OAc, OBn, OR₈, NR₉R₁₀, 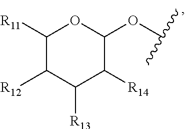
Ph, amino, 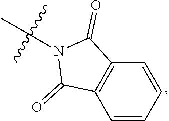 [SMILES: *N1C(=O)c2ccccc2C1=O]
and 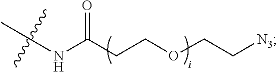
i is an integer selected from 0 to 4;
R₁₁, R₁₂, R₁₃, and R₁₄ are each independently selected from L₂R_{y}; L₂ is selected from absence or methylene, and R_{y} is selected from the group consisting of H, OH, C₁₋₅ alkyl, OAc, OBn, OR₈, and NR₉R₁₀;
R₈ is selected from C₁₋₅ alkyl;
R₉ is selected from the group consisting of H, C₁₋₅ alkyl, Ac, and Bn;
R₁₀ is selected from the group consisting of H, C₁₋₅ alkyl, Ac, and Bn;
M⁺ is a monovalent cation.

5. The glycosyl donor according to claim 3, or salts thereof, or stereoisomers thereof, or optical isomers thereof, characterized in that the 5-6-membered ring is a 5-6-membered saturated oxygen-containing heterocycle;
R₈ is selected from C₁₋₃ alkyl;
R₉ is selected from the group consisting of H, C₁₋₃ alkyl, Ac, and Bn;
R₁₀ is selected from the group consisting of H, C₁₋₃ alkyl, Ac, and Bn;
M⁺ is selected from the group consisting of Na⁺, K⁺, and Li⁺.

6. The glycosyl donor according to claim 1, or salts thereof, or stereoisomers thereof, or optical isomers thereof, characterized in that the structure of the glycosyl donor is selected from the group consisting of:

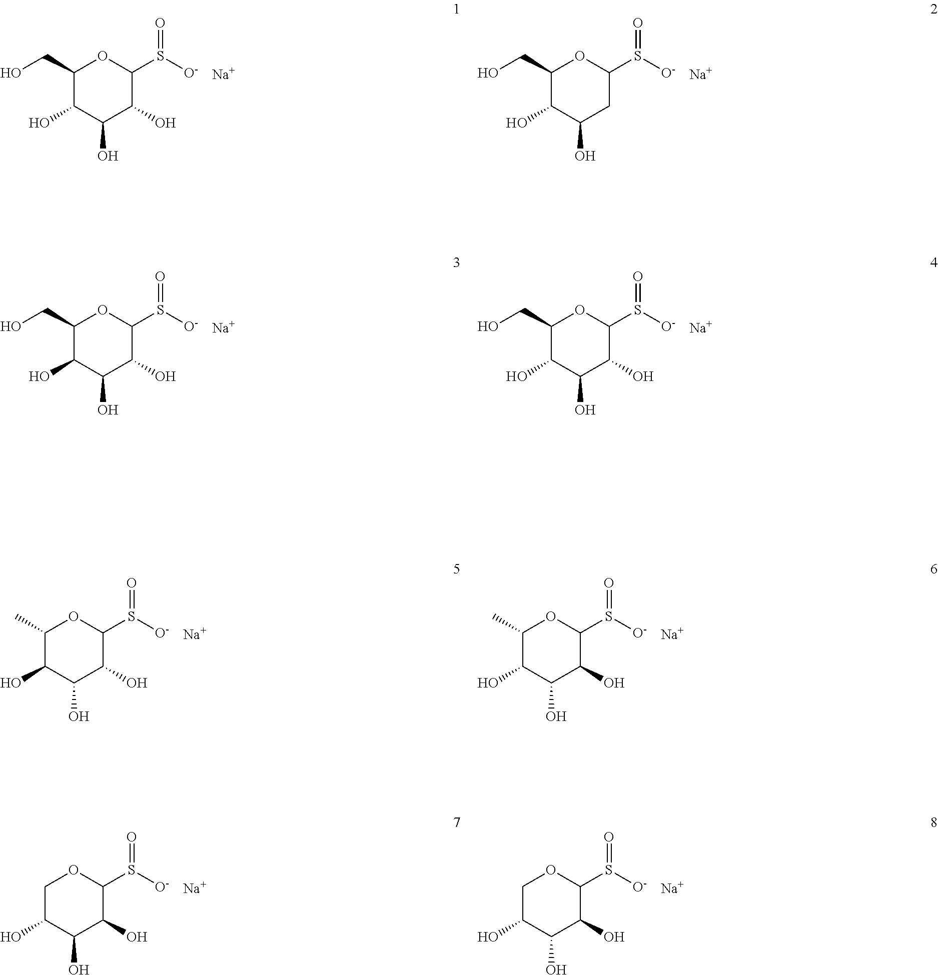

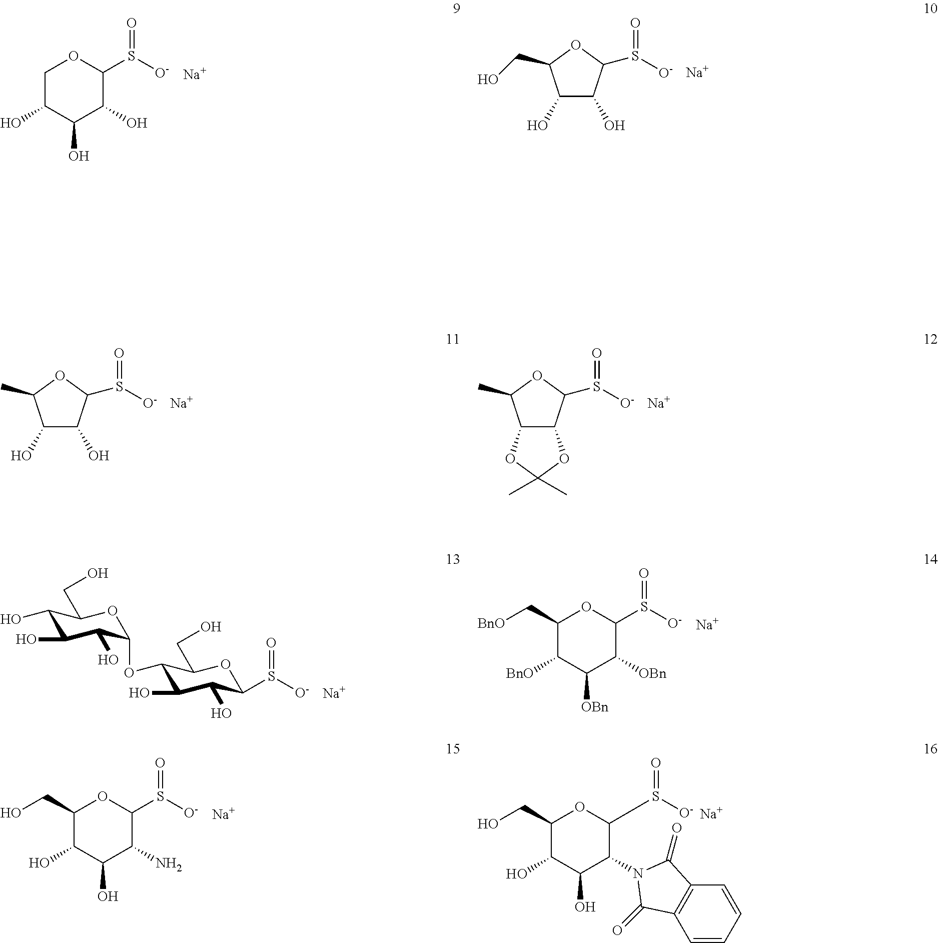

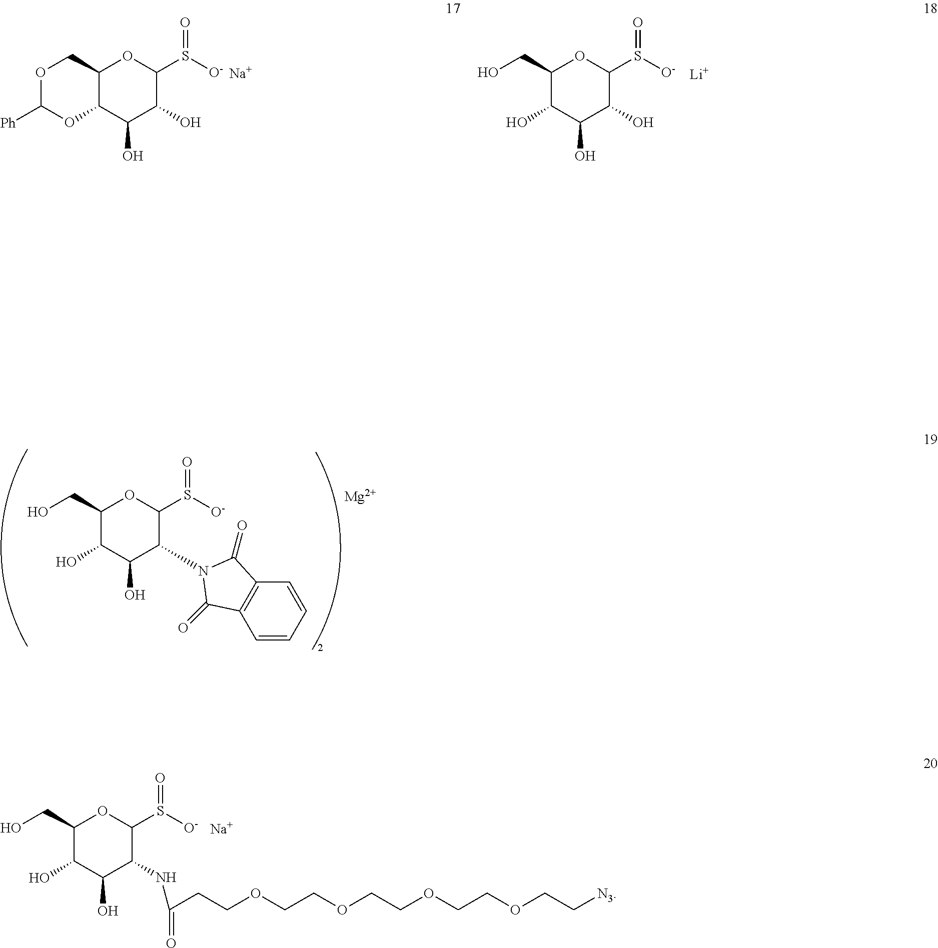

7. A method for preparing the glycosyl donor according to claim 1, characterized in that the method comprises the following steps: 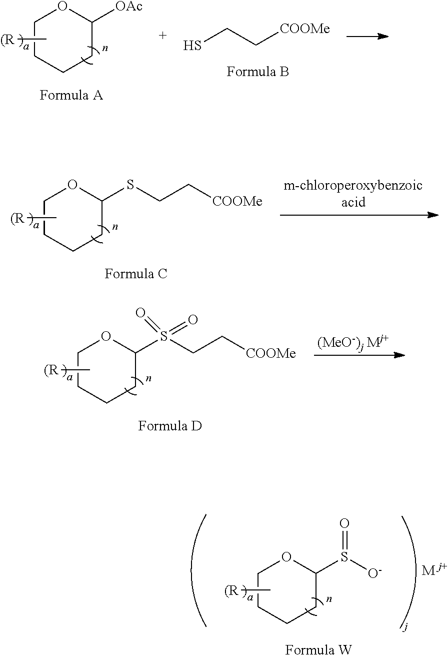
(1) The compound represented by formula A reacts with the compound represented by formula B, to obtain the compound represented by formula C;
(2) The compound represented by formula C reacts with m-chloroperoxybenzoic acid, to obtain the compound represented by formula D;
(3) The compound represented by formula D reacts with (MeO⁻)ⱼM^{j+}, to obtain the compound represented by formula W;
wherein n, a, R, M^{j+}, and j are as defined in claim 1.

8. The method according to claim 7, characterized in that in step (1), the molar ratio of the compound represented by formula A to the compound represented by formula B is 1:(1.0-2.0), the reaction is carried out in the presence of Lewis acid, the molar ratio of the compound represented by formula A to Lewis acid is 1:(1.0-3.0), the reaction solvent is an organic solvent, the reaction temperature is room temperature, and the reaction time is 0.5-3 h;
in step (2), the molar ratio of the compound represented by formula A to m-chloroperoxybenzoic acid is 1:(2-4), the reaction solvent is an organic solvent, the reaction temperature is room temperature, and the reaction time is 0.5-3 h;
in step (3), the molar ratio of the compound represented by formula A to (MeO⁻)ⱼM^{j+} is 1:(0.5-1.5), the reaction solvent is an organic solvent, the reaction temperature is −5° C. to 5° C., and the reaction time is 1-3 h.

9. The method according to claim 8, characterized in that in step (1), the molar ratio of the compound represented by formula A to the compound represented by formula B is 1:1.2, Lewis acid is BF₃·Et₂O, the molar ratio of the compound represented by formula A to Lewis acid is 1:2.0, the organic solvent is dichloromethane, and the reaction time is 1 h;
in step (2), the molar ratio of the compound represented by formula A to m-chloroperoxybenzoic acid is 1:3, the organic solvent is dichloromethane, and the reaction time is 1 h;
in step (3), the molar ratio of the compound represented by formula A to (MeO⁻)ⱼM^{j+} is 1:1, the organic solvent is methanol, the reaction temperature is 0° C., and the reaction time is 2 h.

10. The use of the glycosyl donor according to claim 1 in the preparation of glycoside compounds; the glycoside compound is preferably a S-glycoside compound or a C-glycoside compound.

11. The use according to claim 10, characterized in that the C-glycoside compound comprises aryl C-glycoside compounds.

12. A method for preparing a C-glycoside compound, characterized in that the method comprises the following steps: the glycosyl donor according to claim 1, the glycosyl acceptor represented by formula A-1, and a photosensitizer are added to a solvent, and then allowed to react under light, to obtain the C-glycoside compound represented by formula A-2; 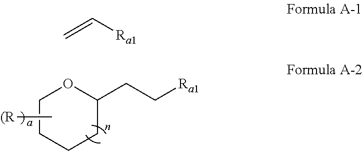
wherein R, a, and n are as defined in claim 1;
Rₐ₁ is selected from the group consisting of H, hydroxyl, C₁₋₆ alkyl, C₁₋₆ alkoxy, halogen, and the following moieties unsubstituted or substituted with one or more Rₐ₂ groups: 5-6-membered aryl, 5-6-membered heteroaryl, 3-8-membered saturated cycloalkyl, 3-8-membered saturated heterocyclyl; Rₐ₂ is selected from the group consisting of hydroxyl, C₁₋₆ alkyl, C₁₋₆ alkoxy, halogen, COORₐ₃, CORₐ₃; Rₐ₃ is selected from H and C₁₋₆ alkyl.

13. The method according to claim 12, characterized in that the molar ratio of the glycosyl donor, the glycosyl acceptor represented by formula A-1, and a photosensitizer is 1:(1-3):(5-20), and preferably 1:1.5:10;
the photosensitizer is selected from the group consisting of Ru (II) photosensitizer, eosin Y or a salt thereof, and said Ru (II) photosensitizer is preferably Ru(bpy)₃Cl₂·6H₂O, and the salt of eosin Y is preferably Eosin Y/Na⁺;
the solvent is an organic solvent; and preferably DMSO, DMF, DMA; and more preferably DMSO;
the reaction is carried out under a nitrogen atmosphere, the reaction temperature is room temperature, and the reaction time is 10-14 h;
said Rₐ₁ is

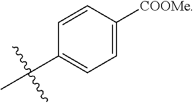 [SMILES: *c1ccc(C(=O)OC)cc1]

14. A method for preparing a S-glycoside compound, characterized in that the method comprises the following steps: the glycosyl donor according to claim 1, the glycosyl acceptor represented by formula C-1, and a photosensitizer are added to a solvent, and then allowed to react under light, to obtain the S-glycoside compound represented by formula C-2; 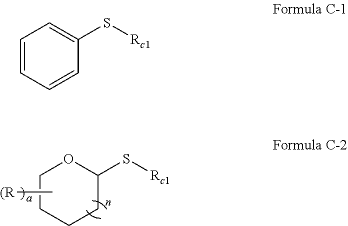
wherein R, a, and n are as defined in claim 1;
R_{c1} is selected from the group consisting of H, hydroxyl, C₁₋₆ alkyl, C₁₋₆ alkoxy, halogen, and the following moieties unsubstituted or substituted with one or more R_{c2} groups: 5-6-membered aryl, 5-6-membered heteroaryl, 3-8-membered saturated cycloalkyl, 3-8-membered saturated heterocyclyl; R_{c2} is selected from the group consisting of hydroxyl, C₁₋₆ alkyl, C₁₋₆ alkoxy, halogen, COOR_{c3}, COR_{c3}; R_{c3} is selected from H and C₁₋₆ alkyl.

15. The method according to claim 14, characterized in that the molar ratio of the glycosyl donor, the glycosyl acceptor represented by formula C-1, and a photosensitizer is 1:(1-3):(5-20), and preferably 1:1.5:10;
the photosensitizer is selected from the group consisting of Ru (II) photosensitizer, eosin Y or a salt thereof, and said Ru (II) photosensitizer is preferably Ru(bpy)₃Cl₂·6H₂O, and the salt of eosin Y is preferably Eosin Y/Na⁺;
the solvent is an organic solvent; and preferably DMSO, DMF, DMA; and more preferably DMSO;
the reaction is carried out under a nitrogen atmosphere, the reaction temperature is room temperature, and the reaction time is 10-14 h;
said R_{c1} is 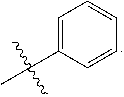 [SMILES: *c1ccccc1]
